(19) United States
(12) Patent Application Publication
(10) Pub. No.: US 2019/0209100 A1
(43) Pub. Date: Jul. 11, 2019

(54) IN-VEHICLE USER HEALTH PLATFORM SYSTEMS AND METHODS

(71) Applicant: Byton Limited, Hong Kong (HK)

(72) Inventors: Petar Strahinja, Shanghai (CN); Henrik Wenders, Shanghai (CN)

(21) Appl. No.: 15/863,755

(22) Filed: Jan. 5, 2018

(51) Int. Cl. A61B 5/00; A61B 5/145; A63B 24/00; A61B 5/021; A61B 5/01; G06F 3/0482

(52) U.S. Cl. CPC A61B 5/7445; G06F 3/0482; A61B 5/4806; A63B 24/0062; A61B 5/6893; A61B 5/486; A61B 5/743; A61B 5/002; A61B 5/0022; A61B 5/681; A61B 5/021; A61B 5/015; A61B 5/7435; A61B 5/7475; A61B 5/0077; A61B 5/14542

(57) ABSTRACT

In-vehicle user health platform systems and methods are disclosed. The in-vehicle health platform system can include a display within a vehicle and a computer coupled to the display. The display can be on a dashboard of the vehicle or display computer having a plurality of display areas. The computer can receive health data for a user and provide options for the user to select a type of health data and report to display in one or more display areas of the display based on the received health data. The health data can include at least one of sleep data, vital signs data, oxygen level data, and workout data of the user. The health data can be received from at least one of a wearable, a health measuring device, and a weight measuring device within the vehicle. The health platform system can provide options including a sleep option to display or report the sleep data, vital signs option to display or report the vital signs data, oxygen level option to display or report the oxygen level data, and workout option to display or report the workout data. The computer can display or report the health data by displaying patterns of the health data for the user related to at least one of the sleep option, vital signs option, oxygen level option and workout option in one or more display areas of the display. The patterns can include one or more graphical elements shown on the display. The computer can provide user advice or tips to improve fitness or well-being in one or more display areas of the display based on the patterns of the health data. The advice or tips can include activity suggestions to achieve health or fitness goals or health measurement levels for the user.

### FIELD

Embodiments of the invention are in the field of data processing including user health data processing. More particularly, embodiments of the invention relate to in-vehicle user health platform systems and methods.

### BACKGROUND

Monitoring health and well-being data such as heart rate, number of miles walked, calories burned, and etc. is part of everyday life. Certain activities such as driving or riding in a vehicle such as an automobile should not prevent one from continuing to monitor health and well-being information, in particular, if driving or riding in a vehicle for long distances or in high traffic. Vehicles thus need a platform to allow users or drivers to continue to monitor health or well-being data which can be presented to a user or driver in meaningful and useful ways as part of the driving or riding experience of a vehicle.

### SUMMARY

In-vehicle user health platform systems and methods are disclosed. For one example, an in-vehicle health platform system includes a display within a vehicle and a computer coupled to the display. The display can be on a dashboard of the vehicle or a display computer having a plurality of display areas. The computer can receive health data for a user and provide options for the user to select a type of health data and report to display in one or more display areas of the display based on the received health data. The health data can include at least one of sleep data, vital signs data, oxygen level data, and workout data of the user. The health data can be received from at least one of a wearable, a health measuring device, and a weight measuring device within the vehicle. The wearable can be worn by the user. The health measuring device can be mounted within the vehicle, and the weight measuring device can include one or more sensors in a seat of the vehicle.

For one example, the in-vehicle health platform system provides options including a sleep option to display or report the sleep data, vital signs option to display or report the vital signs data, oxygen level option to display or report the oxygen level data, and workout option to display or report the workout data. The computer can display patterns of the health data for the user related to at least one of the sleep option, vital signs option, oxygen level option and workout option in one or more display areas of the display. The patterns can include one or more graphical elements shown on the display. The computer can provide health reports giving user advice or tips to improve fitness or well-being in one or more display areas of the display based on the patterns of the health data. The advice or tips can include activity suggestions to achieve health or fitness goals or health measurement levels for the user.

For one example, a computer-implemented method within a vehicle includes collecting health data for a user. Options are provided for the user to select a type of health data and report to display based on the collected health data. The collected health data is processed based on the selected options. The selected type of health data and health report are displayed in one or more display areas of a display within the vehicle based on the processed collected health data. For one example, the health data can be collected from a wearable, a health measuring device, or a weight measuring device related to a user. These devices can collect sleep data, vital signs data, oxygen level data, or workout data related to the user within the vehicle. The health data can be received from the wearable, health measuring device, or weight measuring device via a Bluetooth or WiFi communication protocol.

For one example, one of a sleep option, vital signs option, oxygen level option, and workout option can be selected for the user. Health data or a health report for one of the sleep data, vital signs data, oxygen level data, and workout data based on the selected option for the user can be displayed in one or more display areas of the display on the dashboard of the vehicle. In displaying the health data or report, patterns can be displayed for one the sleep data, vital signs data, oxygen level data, and workout data based on the selected option for the user in one or more display areas of the display on the dashboard of the vehicle. For one example, user advice or tips to improve fitness or well-being based on the selected option for the user can be displayed in one or more display areas of the display on the dashboard of the vehicle. The displayed advice or tips can include suggestions to achieve health or fitness goals or health measurement levels for the user.

For one example, the collected health data can be stored in a database for the user. The database can be located in a secured cloud-based system. The collected health data stored in the database can be downloaded for the user. A health report for the user can be generated from the downloaded health data for the user from the database which is displayed in one or more display areas of the display on the dashboard of the vehicle.

Other methods, systems, and computer-readable mediums for in-vehicle health platforms are described.

### BRIEF DESCRIPTION OF THE DRAWINGS

The appended drawings illustrate examples and are, therefore, exemplary embodiments and not considered to be limiting in scope.

FIG. 1 illustrates one example of an inside view of a vehicle with a dashboard having a health platform.

FIG. 2A illustrates one example of a driver control environment with an in-vehicle health platform.

FIG. 2B illustrates one example of a driver control environment with an in-vehicle health platform interfacing with health wearables and measuring devices.

FIGS. 3A-3C illustrates examples of health data sources for collecting health data.

FIGS. 3D-3E illustrates further examples health data sources by capturing user images and features of a user.

FIG. 4 illustrates one example block diagram of data processing or computing system architecture which can be used for an in-vehicle health platform.

FIG. 5 illustrates one example block diagram of a computing system for the data processing system architecture of FIG. 4.

FIG. 6 illustrates a block diagram of a computing system to provide an in-vehicle health platform.

FIG. 7A illustrates one example of an in-vehicle health platform user interface on a dashboard.

FIG. 7B illustrates one example of multiple level health data processing for the health platform user interface of FIG. 7A.

FIG. 8A illustrates one example of a display area showing rear and side view images.

FIG. 8B illustrates one example of a display area showing user health menu options.

FIG. 8C illustrates one example of a display area showing user health reports.

FIG. 9A illustrates one example of user sleep data provided on the health platform user interface.

FIG. 9B illustrates one example of user vital signs data provided on the health platform user interface.

FIG. 9C illustrates one example of user work out data provided on the health platform user interface.

FIG. 9D illustrates one example of user oxygen level data provided on the health platform user interface.

FIG. 10 illustrates one example of a flow diagram of a process to provide in-vehicle user health data and reporting.

### DETAILED DESCRIPTION

Embodiments and examples are disclosed for providing an in-vehicle health platform. The health platform can collect health data of a user within a vehicle and provide meaningful and useful information to the user as part of a driving or riding experience of the vehicle. Health data such as sleep data, vital signs data, oxygen level data, and workout data of the user can be collected from a wearable worn by the user or health measuring devices outfitted in the vehicle and provided to the health platform within the vehicle. The health platform can display patterns of the collected health data of the user and provide advice or tips to improve health or fitness of the user based on the user health data patterns. In this way, a user can continue to monitor health data and to provide a user with advice and tips to maintain user well-being within the vehicle.

For one example, an in-vehicle health platform system includes a display within a vehicle and a computer coupled to the display. The display can be on a dashboard of the vehicle or a display computer having a plurality of display areas. The computer can receive health data for a user and provide options for the user to select a type of health data and report to display in one or more display areas of the display based on the received health data. The health data can include at least one of sleep data, vital signs data, oxygen level data, and workout data of the user. The health data can be received from at least one of a wearable, a health measuring device, and a weight measuring device within the vehicle. The wearable can be worn by the user. The health measuring device can be mounted within the vehicle, and the weight measuring device can include one or more sensors in a seat of the vehicle.

For one example, the in-vehicle health platform system provides options including a sleep option to display or report sleep data, vital signs option to display or report vital signs data, oxygen level option to display or report oxygen level data, and workout option to display or report workout data. The computer can display or report the health data by displaying patterns of the health data for the user related to at least one of the sleep option, vital signs option, oxygen level option and workout option in one or more display areas of the display. The patterns can include one or more graphical elements shown on the display. The computer can provide user advice or tips to improve fitness or well-being in one or more display areas of the display based on the patterns of the health data. The advice or tips can include activity suggestions to achieve health or fitness goals or health measurement levels for the user.

Although the following examples and embodiments are directed to providing an in-vehicle health platform, the health platform can be implemented for any type of vehicles such as automobiles (including electric, non-electric, or self-driving automobiles), aircraft, and marine vehicles with dashboards having display panels and controls. In the following examples and embodiments, a health platform can be used with one or more displays on a dashboard of vehicle or any computing device having a display within a vehicle. Furthermore, as set forth herein, various embodiments, examples and aspects will be described with reference to details discussed below, and the accompanying drawings will illustrate various embodiments and examples. The following description and drawings are illustrative and are not to be construed as limiting. Numerous specific details are described to provide a thorough understanding of various embodiments and examples. However, in certain instances, well-known or conventional details are not described in order to provide a concise discussion of the embodiments and examples.

### Exemplary Vehicle for a Health Platform

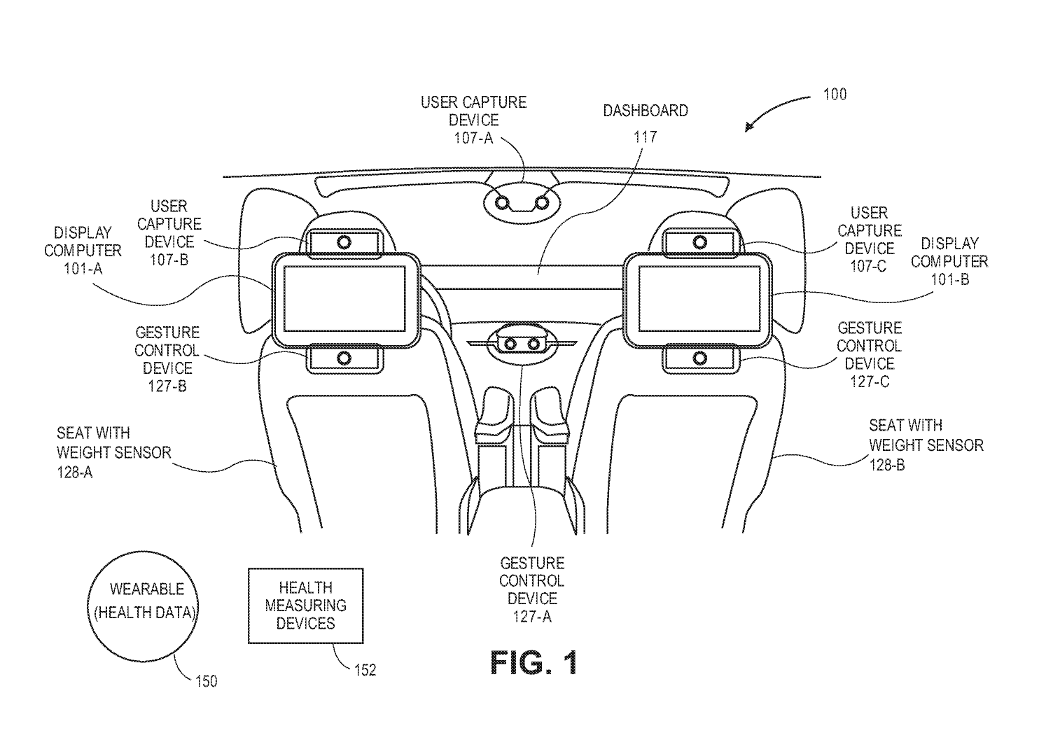

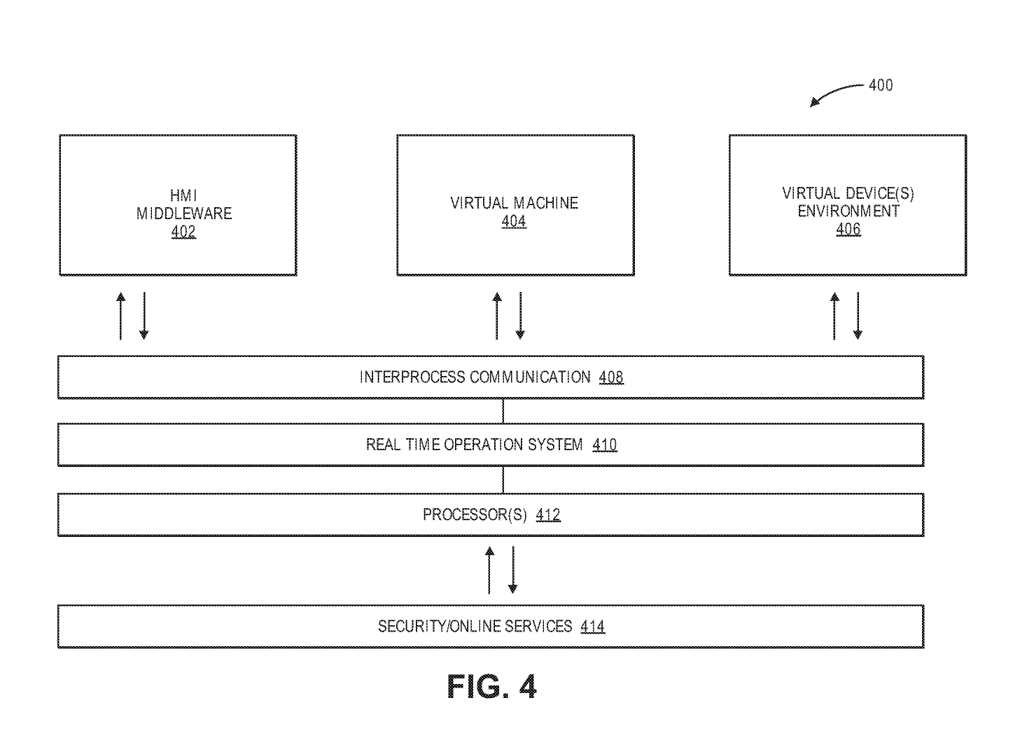

FIG. 1 illustrates one example of an inside view of vehicle 100 from a backset perspective towards dashboard 117 which can have one or more displays and display computers 101-A and 101-B. Dashboard 117 and display computers 101-A and 101-B can provide an interface to a health platform. Vehicle 100 can be an automobile such as an electric or non-electric car or a self-driving or autonomous car. The health platform can allow users, passengers or drivers of vehicle 100 to monitor and evaluate health and well-being data and information during the riding or driving of vehicle 100. The health platform can include one or more computers, computing systems, or data processing systems and other controls as disclosed in FIGS. 4-6. The systems and controls can be coupled to dashboard 117 and display computers 101-A and 101-B for collecting or receiving health data from one or more sources. For example, a wearable 150, health measuring devices 152, or weight sensors in seats 128-8 and 128-B within vehicle 100 can provide health data to the health platform. For example, wearable 150 worn by a user or driver of vehicle 100 can be a smart watch or a fitness watch that can send heart rate data, sleep data, distance walked data, calories burned data, etc. to the health platform. Health measuring devices 152 can include, e.g., a blood pressure measuring device or a finger oxygen level measuring device situated within vehicle 100, e.g., mounted on seat 128-A or 128-B or passenger seats, that can send blood pressure and oxygen level data to the health platform. Seats 128-A and 128-B or passenger seats can include weight sensors to measure the weight of user or driver of vehicle 100 to the health platform.

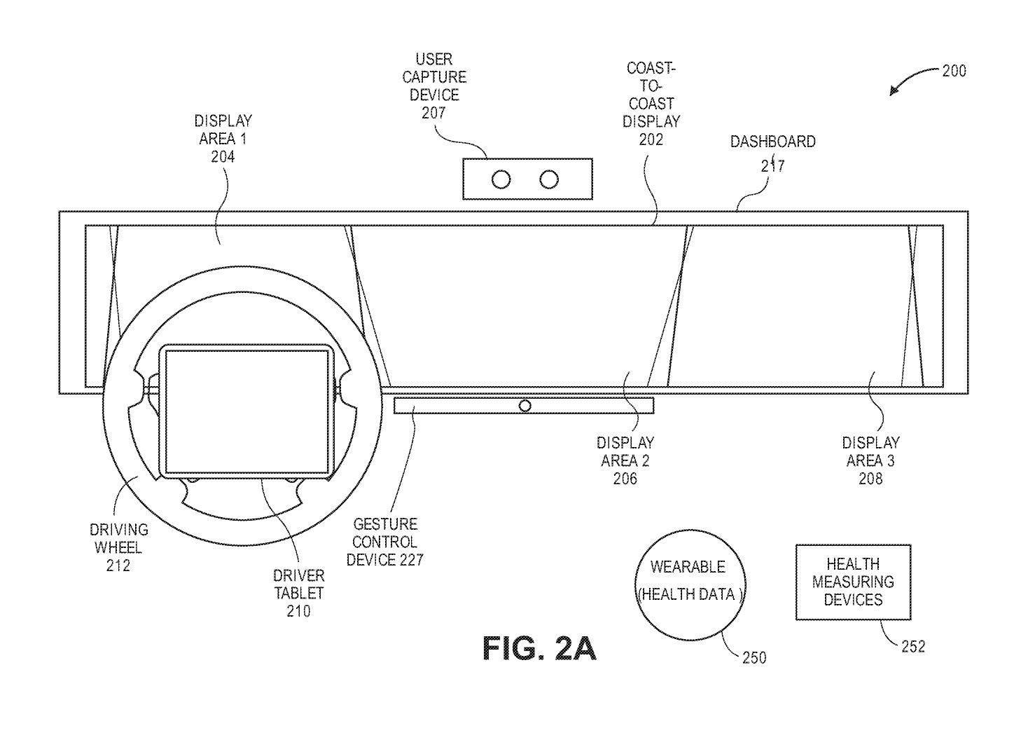

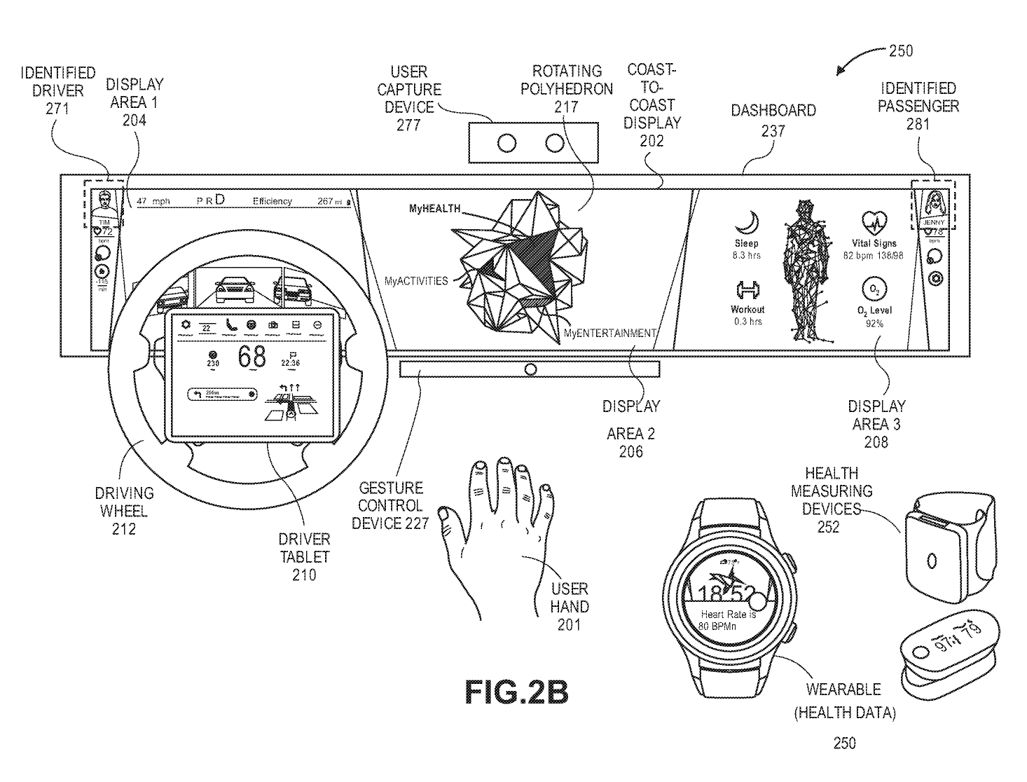

For one example, dashboard 117 includes one or more displays or display areas to provide a user-interface to the health platform. The same or similar interface can also be provided on display computers 1001-A and 101-B. For one example, the interface for the health platform can display patterns of the received or collected health data and display reports giving advice or tips based on the patterns of the health data to users or drivers of vehicle 100 as disclosed in FIGS. 2B and 7A-9D. Health data for users or drivers of vehicle 100 can be stored in one or more databases, which can be downloaded or retrieved by the health platform and can be used to generate patterns and reports of health data for users or drivers of vehicle 100. Examples of types of health data collected or received by the health platform can include sleep data (e.g., how many hours slept per day), vital signs data (e.g., heart rate beats per minute, blood pressure systolic/diastolic, weight), oxygen O₂ level data (e.g., blood oxygen saturation levels), and workout data (e.g., distance walked, calories burned, and etc.).

Referring to FIG. 1, vehicle 100 can include other devices to interface with the health platform and to monitor health and well-being of a user or driver of vehicle 100. For one example, vehicle 100 includes a plurality of user capture devices 107-A through 107-C and gesture control devices 127-A through 127-C. User capture device 107-A can be located above dashboard 117 at the top of the front windshield. User capture devices 107-B and 107-C can be located on the backside of the driver and passenger seat head rests. Each user capture devices 107-A through 107-C can include one or more stereo, RGB (red, green, blue), or infrared cameras to capture user images (e.g., user facial images, expressions, and features) or thermal differential information (e.g., temperature differential information of a user head and surrounding area).

For one example, user capture device 107-A can capture one or more images or expressions of a user (e.g., expressions of the driver or passenger) such as, e.g., a selfie, smile, frown, sleeping, dozing, eyes opening and shutting, anger, happiness, fatigue, anger, stress, or shaking. For one example, computing systems and control for automobile 110 can react to a captured user expression, e.g., if falling sleep providing an audio alert or if recognizing annoyed expression if music is too loud lowering the volume of the radio. Computing systems and controls for vehicle 100 can be configured or programmed to react in any desired manner and differently for a user or driver of vehicle 100 based on images from capture device 107-A or wearable 150, health measuring devices 152, or seats 128-A and 128-B with weight sensors. For example, if no heartbeat is being detected of a driver of vehicle wearing wearable 150, the computing systems and controls for vehicle 100 can be programmed to safely stop vehicle 100. For another example, if seat 128-A with weight sensor detects that no one is sitting in the driver seat of vehicle 100, computing systems and controls for vehicle 100 can be programmed to enter autonomous driving mode.

For one example, user capture devices 107-B and 107-C on the back of head rests for seats 128-A and 128-B can operate in the same way as user capture device 107-A for passengers behind the driver set and front passenger seat and settings and preferences for display computers 101-A and 101-B can also be set accordingly to settings and preferences for a recognized user in the back seats. For one example, user capture devices 107-A through 107-C can include an infrared camera and detect temperature differential information of users in determining wellness, e.g., if a user is sick with a fever.

For one example, vehicle 100 includes gesture control devices 127-A through 127-C located below a dashboard of vehicle 100 and display computers 101-A and 101-B. Each of these gesture control devices 127-A through 127-C can include one or more cameras (e.g., time of flight TOF cameras) or motion sensors to detect hand gestures and movement of a user (e.g., a user, driver or passenger of vehicle 100) in controlling or accessing functions, applications, information, options, icons, or objects provided on a display of the dashboard 117 or display computers 101-A and 101-B. For one example, gesture control devices 127-A through 127-C can include hardware and software from Intel Realsense® to implement gesture control of interfaces on a display of dashboard 137 or display computers 151-A and 151-B.

### Exemplary in-Vehicle Health Platform Interfaces

FIGS. 2A-2B illustrate exemplary user or driver control environments 200 and 250 for a vehicle (e.g., vehicle 100) proving an interface to a health platform. Referring to FIG. 2A, a driver control environment 200 is shown from a front seat view perspective. For one example, environment 200 includes vehicle dashboard 217 with a driving wheel 212 having a driver tablet 210 and coast-to-coast display 202. Coast-to-coast display 202 includes three display areas: display area 1 (204), 2 (206) and 3 (208). Although not shown, one more computing devices (computers) can be situated behind or within dashboard 217, which can provide automobile controls and applications including health platform applications. For one example, user capture device 207 can be mounted and located above dashboard 217 and includes one or more cameras to capture images or expressions of users (e.g., the user, driver or passenger in the front seats). Gesture control device 227 can be mounted below coast-to-coast display 202 on dashboard 217. Gesture control device 227 can include or more motion cameras (e.g., time of flight TOF cameras) and sensors to capture, e.g., hand gestures of a user, to access and control applications and interfaces on display areas 1-3 (204, 206, and 208) of coast-to-coast display 202.

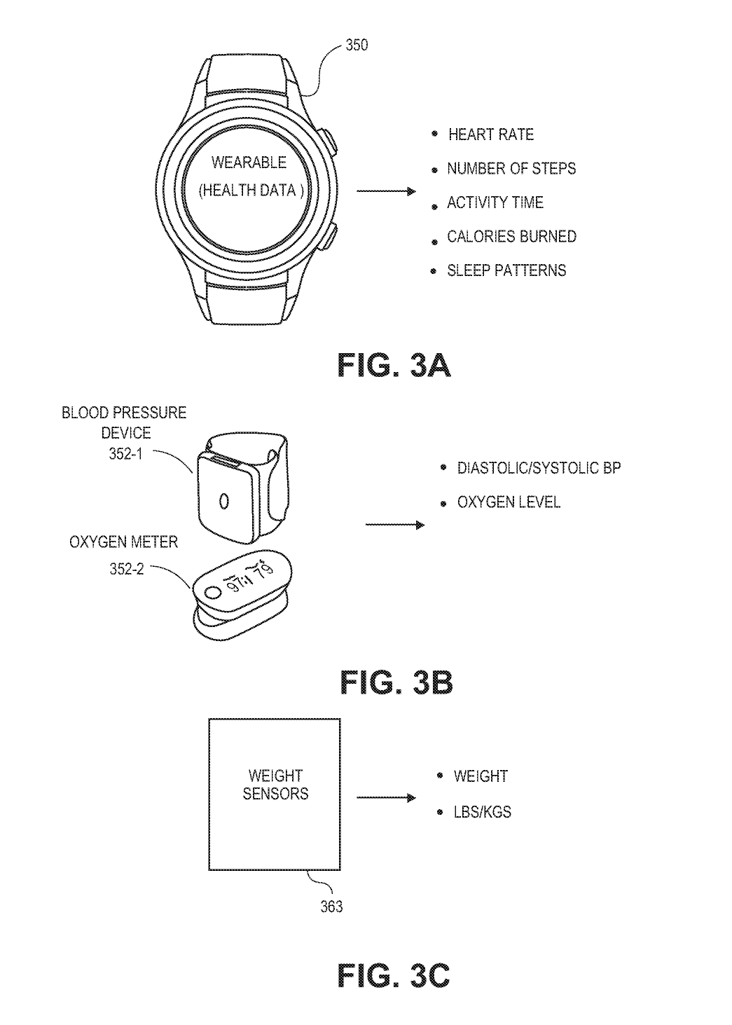

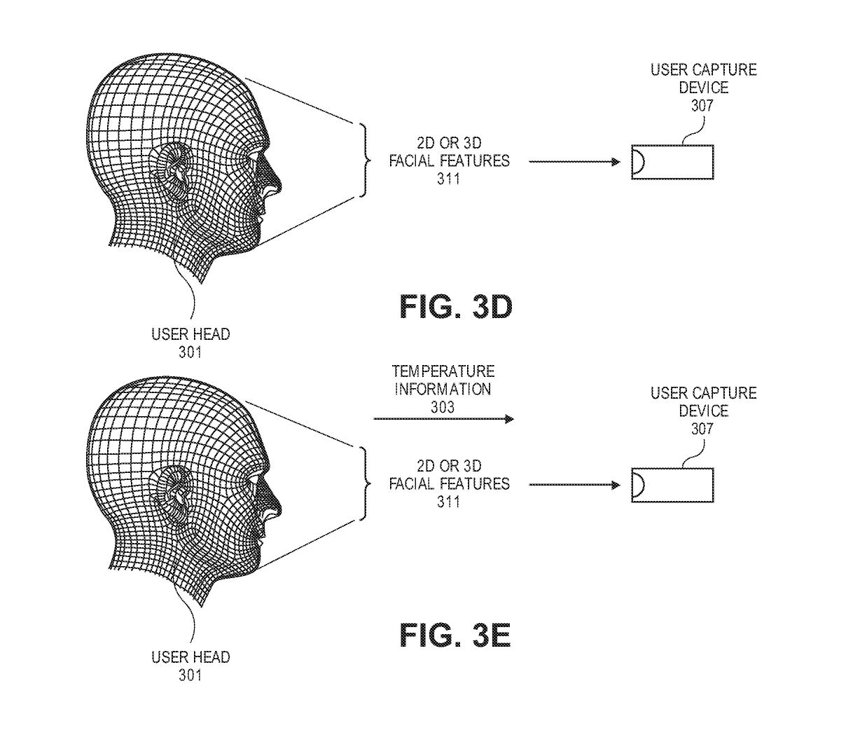

For one example, user capture device 207 can be mounted above dashboard 217 e.g., on a windshield of the vehicle and can include one or more cameras and operate in the same way as user capture devices as described in FIGS. 3D-3E to capture user images, features and expressions. The location of user capture device 207 can be located or mounted so as to be aligned to capture user images and expressions of a user or driver or front seat passenger. Other locations for user capture device 207 can be on dashboard 217, a pillar left of driving wheel 212 (inside or outside of the automobile), a pillar right of passenger seat (inside or outside of vehicle 100). Although not shown, other cameras can be mounted within or outside of the automobile to take side view and rearview images which can be shown on coast-to-coast display 202, e.g., on left side in display area 1 (204) to avoid having to use side view mirrors and a rearview mirror.

Referring to FIG. 2B, vehicle environment 250 is shown from front view perspective with user interfaces and applications provided on coast-to-coast display 202 on dashboard 217. Driving wheel 212 and driver tablet 210 are attached to dashboard 217. Driver tablet 210 can provide a driver interface to access controls including settings and preferences for the vehicle (e.g., vehicle 100). For one example, user capture device 277 can be located and positioned above dashboard 237 having one or more cameras to detect and identify a driver (e.g., identified driver 271 “Tim” or identified passenger 281 “Jenny”). For one example, Tim can be authenticated as a valid user or driver using user capture device 277 (e.g., matching user images of Tim with stored user images in a database to detect Tim as a valid user or driver of vehicle 100). User capture device 277 can also capture expressions of a driver or passenger, e.g., Tim or Jenny, and provide a reaction, e.g., an audio alert if a driver is falling asleep. For other examples, if a child is sitting in the driver seat, user capture device 237 can capture an image of the child and driver tablet 210 or an automobile computer can detect the child and deny controls to driver tablet 210 or coast-to-coast display 202 and prevent the child from driving the automobile or accessing any of its controls.

For one example, once authenticated as a valid driver, e.g., Tim, driver tablet 210 or an automobile computer (e.g., behind or within dashboard 237) can configure settings and preferences for Tim including settings and preferences for control interfaces on coast-to-coast display 202. For example, as shown in display area 3 (208), MyHealth platform settings may be set for Tim with options related to health data for Sleep, Workout, Vital Signs, and Oxygen O₂ level as shown in display area 3 (208) and a corresponding rotating polyhedron 217 for Tim to select the MyHealth platform in which other options include MyActivities and MyEntertainment can also be selected using rotating polyhedron 217 as a graphical control object. Furthermore, a passenger, e.g., Jenny, can also have settings and preferences set designated for Jenny on coast-to-coast display 202 once authenticated. Examples of settings and preferences can include personalized user interfaces on coast-to-coast display 202, personalized health platform, personalized seat controls, personalized steering wheel controls, pedal locations, personalized climate control, personalized phone interface, personalized mapping etc.

For one example, gesture control device 227 is located and positioned below automobile dashboard 237 having one or more cameras and motion sensors to detect hand gestures and movement of user hand 201. For example, user hand 201 can represent a hand of a user, driver or a passenger (e.g., who have been properly recognized as a valid user) and gesture control device 227 can capture user gestures (e.g., gestures of user hand 201) in controlling or accessing functions, applications, information, options, icons, or objects provided on coast-to-coast display 202 including MyHealth platform options in display areas 2 and 3 (206 and 208). For one example, gesture control device 227 can include hardware and software from Intel Realsense® and user capture device 277 can include one or more cameras, e.g., a time of flight TOF camera.

For one example, driver tablet 210 is a tablet computer and can provide a touch screen with haptic feedback and controls. Driver tablet 210 can provide primary vehicle function controls for a user or driver such as climate control and various settings for environment 250 which can be set once a valid user is identified and authenticated, e.g., identified driver 271 Tim or identified passenger 281 Jenny. Driver tablet 210 can be coupled to a vehicle computer (not shown) behind or within dashboard 237, user capture device 277 or gesture control device 227. Driver tablet 210, vehicle computer, or both can be configured to recognize a driver (e.g., Tim) or a passenger (e.g., Jenny) and allow the user, driver or passenger to use gesture control device 227 and access coast-to-coast display 202. For one example, driver tablet 210 can provide any number of representations, objects, icons, or buttons on its touchscreen providing functions, navigation user interface, phone control user interface, and interfaces to coast-to-coast display 202. Driver tablet 210 can also be paired with wearable 250 and health measuring devices 252 via a Bluetooth connection and protocol. Additionally, one or more computers or computing systems behind or within dashboard 237 can be paired with wearable 250 and health measuring devices 252 via a wireless connection or protocol, e.g., Bluetooth or WiFi connection and protocol, or other Bluetooth or WiFi devices such as a mobile phone. For example, one or more vehicle 100 computers can collect and receive health data from wearable 250 and health measuring devices 252. For one example, settings and pairings for wearable 250 and health measuring devices 252 or other wireless devices such as a mobile phone can establish a secured connection with one or more computers behind or within dashboard 237 via a Bluetooth pairing protocol. The settings and pairings for wearable 250 and health measuring devices 252 can include device data such that wearable 250 and health measuring devices 252 can be discoverable by vehicle health platform computer(s).

For one example, wearable 250 can be a smartwatch or a fitness watch with a Bluetooth connection providing health data such as sleep data (e.g., how many hours slept per day), vital signs data (e.g., heart rate beats per minute), and workout data (e.g., distance walked, calories burned, and etc.) to the vehicle 100 health platform computer(s). Health measuring devices 252 can include a blood pressure measuring device and an oxygen O₂ level device. The blood pressure measuring device can provide blood pressure systolic/diastolic data and the oxygen level measuring device can provide blood oxygen saturation levels. Other devices such as weight sensors on seats within vehicle 100 can provide weight data via a Bluetooth connection or by wired connection to vehicle health platform computer(s).

Coast-to-coast display 202 can include a light emitting diode (LED) display, liquid crystal display (LCD), organic light emitting diode (OLED), or quantum dot display, which can run from one side to the other side of dashboard 237. For one example, coast-to-display 202 can be a curved display integrated into and spans the width of dashboard 237. One or more graphical user interfaces can be provided in a plurality of display areas such as display areas 1 (204), 2 (206), and 3 (208) of coast-to-coast display 202. Such graphical user interfaces can include status menus, application menus, options and settings, data and information, and rotating polyhedron 217 for a user to access different applications MyHealth, MyActivities, and MyEntertainment, as shown in, e.g., display areas 1 (204), 2 (206) and 3 (208).

For one example, display area 1 (204) can show rearview or side view images of the vehicle from one or more cameras which can be located outside or inside of the vehicle in order to capture rear view of side view images. For one example, display area 2 (206) can provide and display a rotatable three-dimensional object such as rotating polyhedron 217 having polygonal faces defined by dots and lines. Alternatively, display area 3 (208) can display rotating polyhedron 217. Rotating polyhedron 217 can appear in display area 2 (206) as floating in space and can rotate at a constant or variable speed having one or faces designated to an application such a “MyHealth”, “MyActivities”, and “MyEntertainment” and when selected by a user can provide one or more interfaces on coast-to-coast display 202.

For one example, rotating polyhedron 217 can provide a group of information using one or more faces, dots, and lines which can provide a tangible form of various parameters and types of user information for each user (driver, or passenger) which has been authenticated as valid user. That is, each authenticated or valid user of the vehicle can have a personalized rotating polyhedron 217 for accessing and controlling user information and applications shown display area 2 (206) and display area 3 (208). For example, any number of drivers or users can be registered with stored bio-scan information such as, e.g., facial features or expressions, in a database. User capture device 277 can be used to identify, recognize, and authenticate as a user as valid and driver tablet 210 can provide a personalized polyhedron 217 for each valid user.

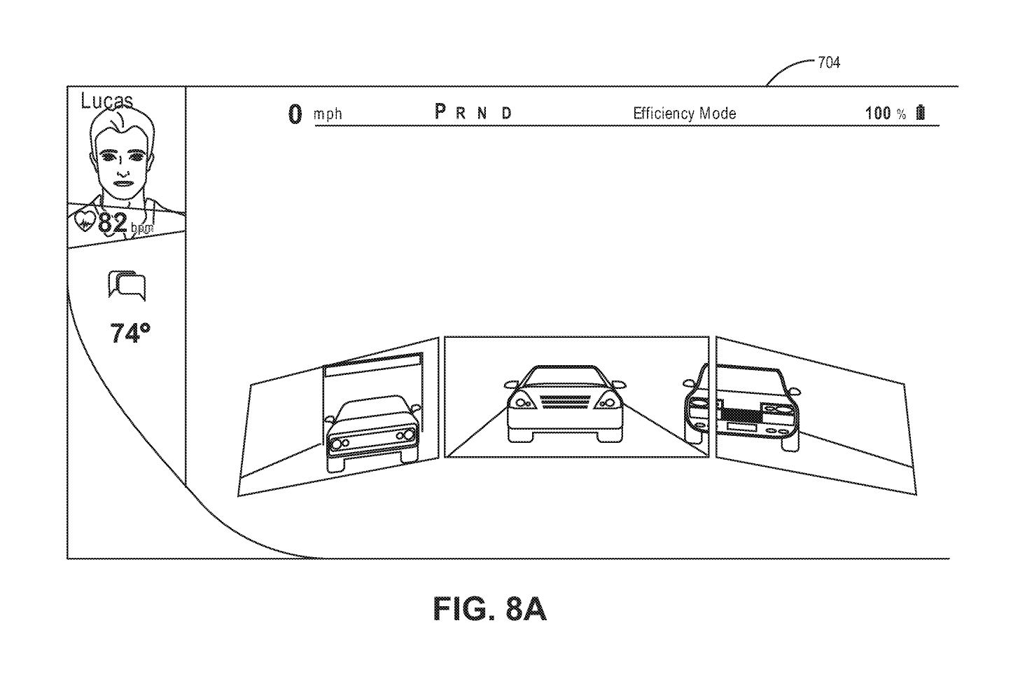

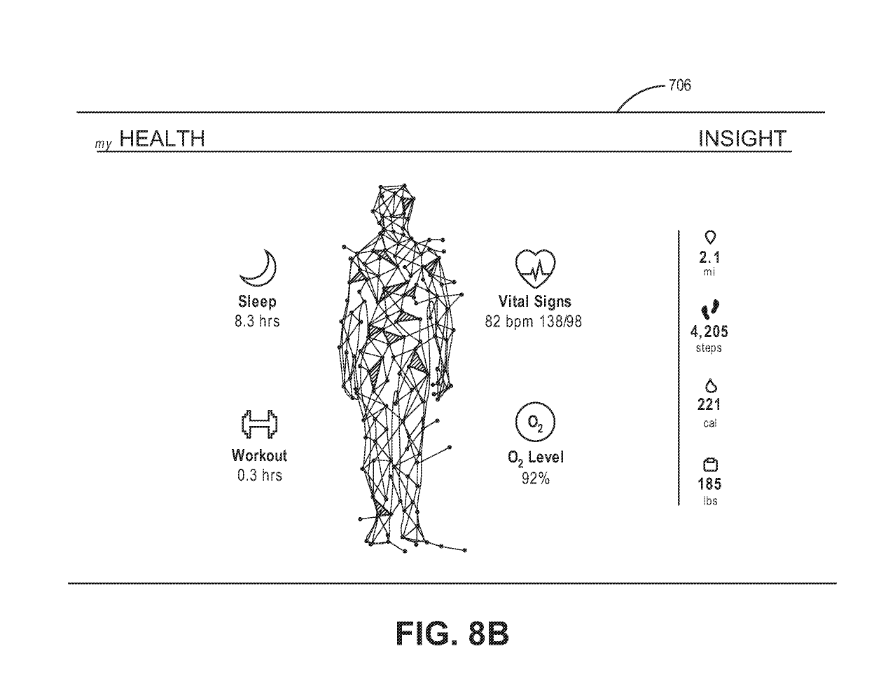

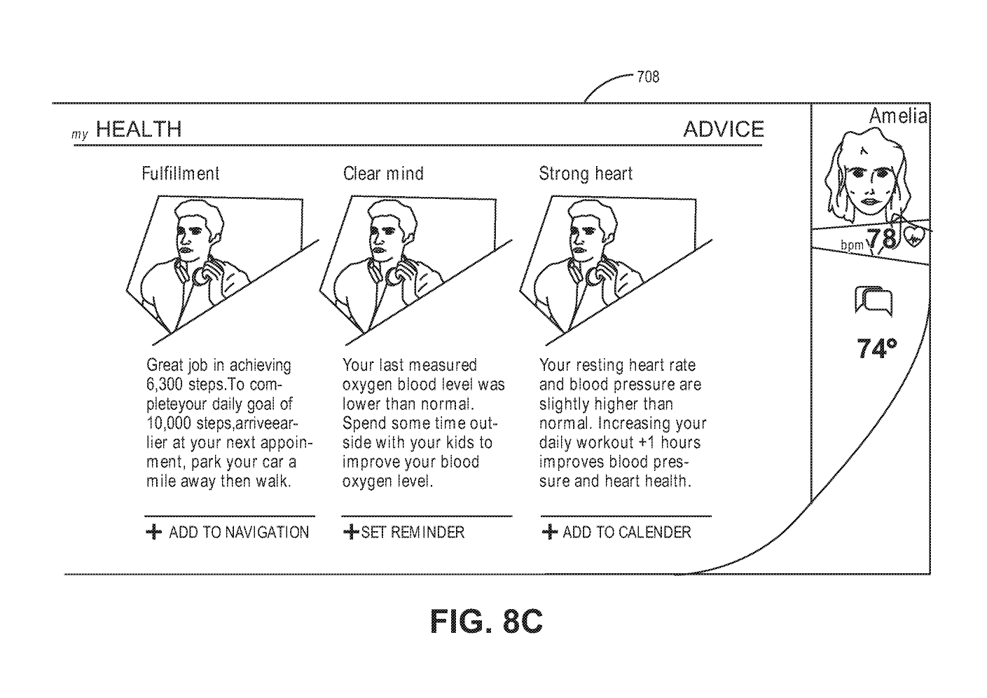

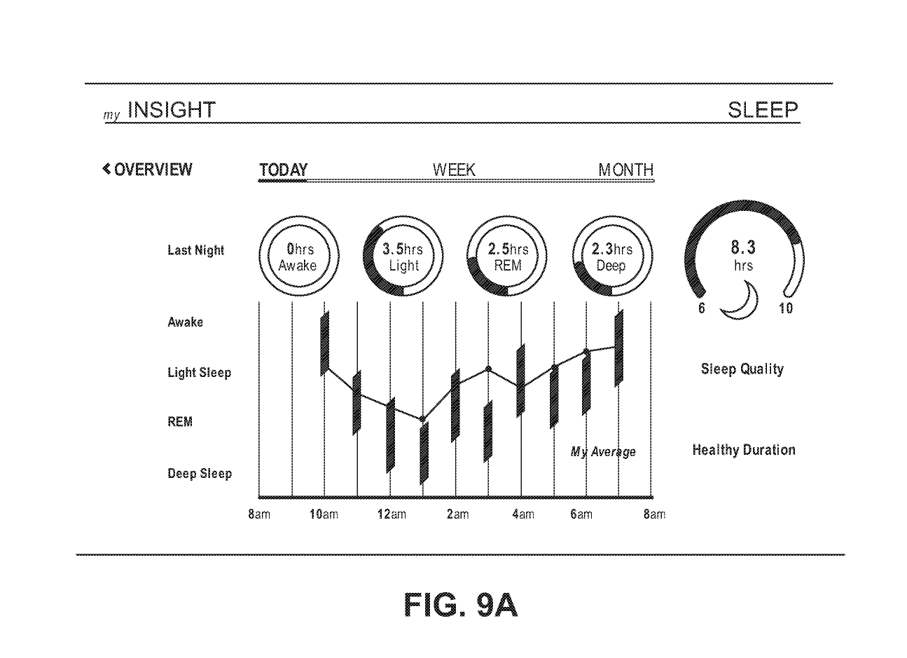

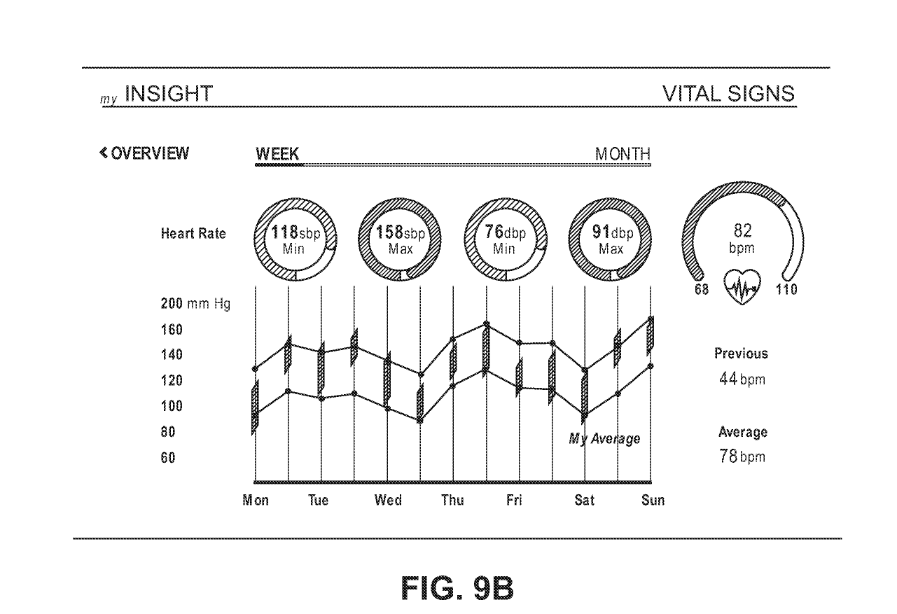

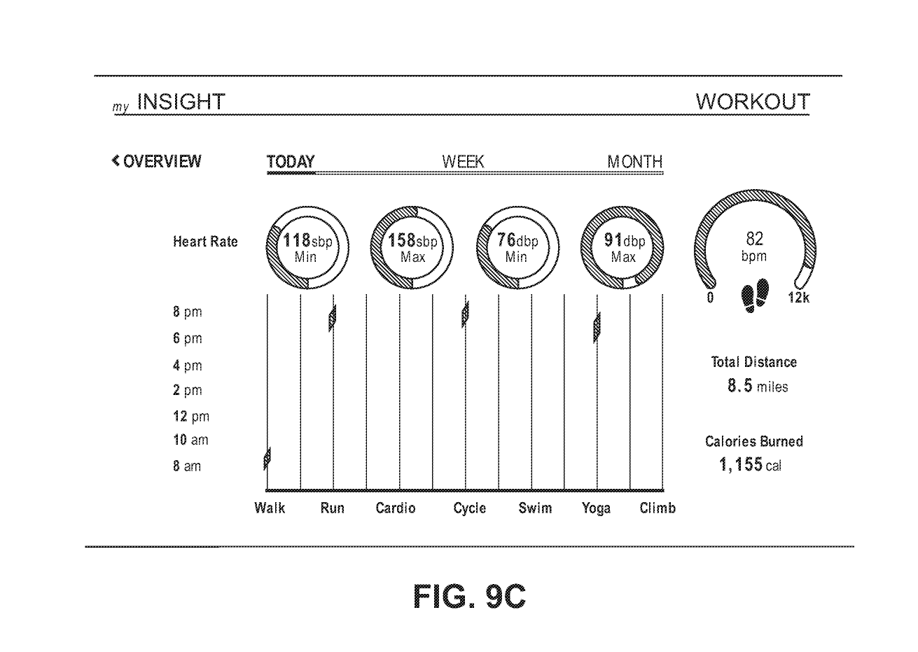

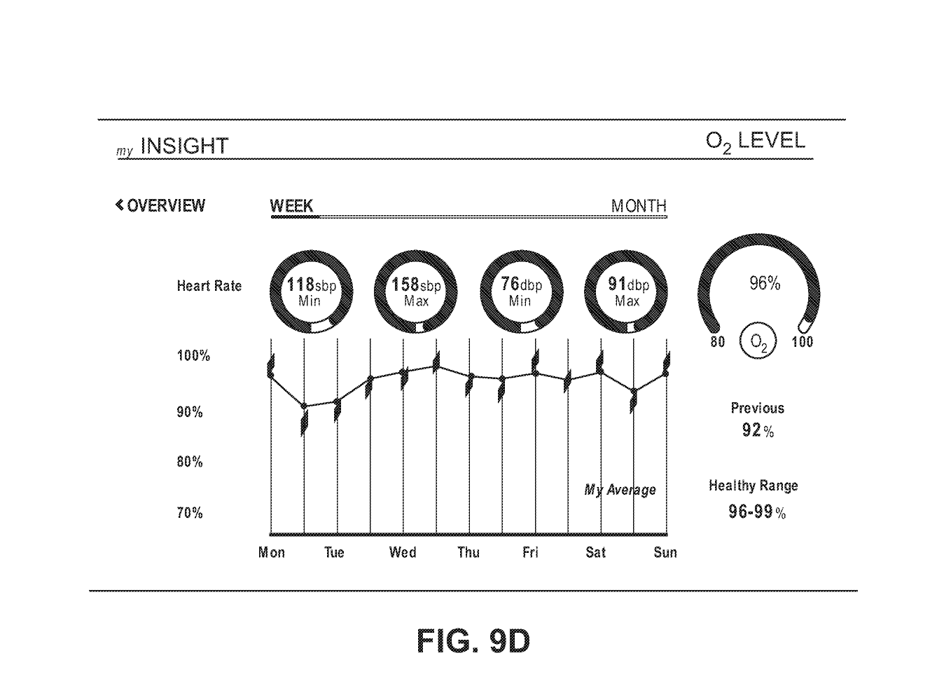

Examples of types or groups of information or applications which can be associated and personalized for each valid user (e.g., a valid driver or passenger) can include user information and applications, e.g., MyHealth, MyEntertainment, and MyActivities, with a corresponding face on rotating polyhedron 217 as shown in display area 2 (206). The dots or lines and number of dots and lines defining polygonal faces on rotating polyhedron 217 can also represent various parameters related to user information such as, e.g., MyHealth, which has four dots on the face of the polyhedron corresponding to user health menu having four categories or options such as “Sleep”, “Vital Signs”, “Workout” and “O₂ level” for a user to select as shown in display area 3 (208) and in FIGS. 8A-9D. For one example, the number of dots and lines can alter and modify the shape of rotating polyhedron 217. For example, if more dots are health related, the polygonal face for “MyHealth” can have polygonal surface with a larger number of dots and lines with a larger face.

For one example, a driver or user hand 201 can rotate polyhedron 217 along any axis using hand gestures captured by gesture control device 227 to select a user information or application by moving a desired face of the polyhedron 227 to the foreground, e.g., the foreground of display area 2 (206). Referring to FIG. 2A, MyHealth is in the foreground indicating that it is a selected user information or application. For one example, when a selected user information or application is positioned in the foreground, e.g., MyHealth, by user hand 201 the user information or application icons, categories, items, controls, etc. are shown in display area 3 (208). For other examples, a control object or cursor or avatar can be shown in coast-to-coast display 202 to select faces on polyhedron 2177. Examples of user gestures to rotate the polyhedron include moving the hand or fingers from left to right or vice versa to rotate the polyhedron 217 accordingly. Other movements can be recognized to rotate polyhedron 217 along different axis to move a desired face of polyhedron 217 to the foreground to select the desired user information or application, e.g., MyHealth. Once a desired face of polyhedron 217 is in the foreground, a user can provide a grab and release motion with user hand 201 to obtain additional information regarding the selected user information or application.

For one example, a user or driver can also rotate rotating polyhedron to place MyEntertainment or MyEntertainment in the foreground to select that category or option. The polygonal face for MyEntertainment has four dots or sides which can correspond to four parameters or categories, e.g., Music, Audiobooks, Movies, and Games, to be shown in display area 3 (208). A driver or user can motion with user hand 201 to select one of the options in display area 3 (208) captured by gesture control device 227 to access, e.g., a particular music item in display area 3 (308) under Music category to play. MyActivities also has four points and lines which can represent four different parameters or categories of user information related to MyActivities. For one example, each driver or user can have a user account which can generate a minimum number of baseline dots in rendering rotating polyhedron 217 on coast-to-coast display 202. The driver or user can also add dots on specific types of information to track, e.g., missed calls or text messages.

For one example, categories, associated information and parameters can be generated or inputted by a user with driver tablet 210 or downloaded or entered using, e.g., a mobile phone or wearable 213, to driver tablet 210 which controls and provides information to coast-to-coast display 202. For one example, a user or driver is authenticated or identified before information and parameters can be generated or inputted for rotating polyhedron 217, which can be stored in one or more memories or databases stored in or coupled with driver tablet 210. For each user or driver, a personalized rotating polyhedron 217 can be provided and associated with respective personal information and parameters, e.g., heartbeats, heart rate, etc. For example, each user of driver can generate data points or can be automatically generated which can alter the shape of rotating polyhedron 217. The examples and embodiments for using rotating polyhedron 217 can be used any display interface environment such as a display interface for desktops, laptops, tablets, netbooks, mobile phones and devices in reducing clutter on a display.

### Exemplary Health Data and Sources

FIGS. 3A-3C illustrates examples of health data sources for collecting health data. Referring to FIG. 3A, wearable 350 can be a smart watch or fitness watch configured to monitor and provide health data to one or more computers of the health platform in vehicle 100. Wearable 350 can provide heart rate data such as a heart rate of beats per minute (BPM) for a user or driver of vehicle 100 and using control environments of FIGS. 1-2B. Heart rate data can include date and time rate is taken and provide average heart rate over a period of time which can be selected by the user. Wearable 350 can also monitor a physical activity such as walking or running, e.g., number of steps taken during walk or run, time or duration of walk or run, calories burned during activity, etc. Wearable 350 can also have one or more applications that can be used to monitor sleep and provide sleep pattern data. For other examples, sleep pattern data can be collected from could-based databases which can store sleep data from one or more sleep breathing machines which may be used by a user of vehicle 100 and stored in a secure-cloud based environment. For one example, wearable 350 can provide monitored health data to one or more computers of the health platform in vehicle 100 using standard Bluetooth or WiFi communication interfaces and protocols. For one example, wearable 350 can be Bluetooth paired with the computer(s) of vehicle 100 which communicate with wearable 350 using an open application program interface (API) for Bluetooth devices. Computer(s) for vehicle 100 providing a health platform can receive and collect health data from wearable 350 using the API interface for the health platform disclosed herein.

Referring to FIG. 3B, blood pressure device 352-1 and finger oxygen meter 352-1 can be situated or attached to a seat in vehicle 100, e.g., seat 128-A and 128-B. For one example, blood pressure device 352-1 can measure the diastolic/systolic blood pressure of a user and communicate health data to the health platform of vehicle 100 wirelessly using Bluetooth or WiFi communication interfaces and protocols. Oxygen meter 352-2 can measure oxygen levels using a finger of a user such as blood oxygen saturation levels wirelessly using Bluetooth or WiFi communication methods. Blood pressure device 352-1 and oxygen meter 352-2 can communicate with one or more computers of vehicle 100 implementing health platform using API interfaces in the same way as wearable 350. Referring to FIG. 3C, weight sensors 343 can be mounted in seats 128-A and 128-B and measure weight of users or passengers in seats of vehicle 100 providing weight data (e.g., pounds LBS or mass KGS) to one or more health platform computers within vehicle 100.

FIGS. 3D-3E illustrates further examples health data sources by capturing user images and features of a user with user capture device 307. Referring to FIG. 3D, a user head 301 is depicted three-dimensionally. For one example, user capture device 307 can capture two-dimensional (2D) or three-dimensional (3D) images including facial features 311 of user head 301. User head 301 is depicted to illustrate a head of a user, which can be a driver or a passenger for automobile 110, and identified or recognized by user capture device 307. User capture device 307 can include on or more cameras in the same way as user capture devices 107-A through 107-C shown in FIG. 1 or user capture devices 202 or 277 shown in FIGS. 2A and 2B.

For one example, user capture device 307 can include any type of RGB camera to capture 2D user features (e.g., 2D facial features 311) or a stereo camera to capture 3D user features (e.g., 3D facial features 311) of user head 301. Examples of facial features in user images can include shape, size, and dimensions of, e.g., the nose, mouth, ears, eyes, eyebrows, lips, cheeks, birth marks, face width, head size, hair color, etc. of user head 301. For one example, 2D or 3D user images and facial feature information can be stored during registration of bio-scan information for a user of vehicle 100 which can be stored in a secured database within vehicle 100 or remotely in a secured cloud environment. For one example, user capture device 307 can be used to identify and recognize facial features of user head 301 to determine if such features match those stored in the database for the user in authenticating the user as a valid user for vehicle 100. Any type of facial feature recognition algorithms can be used such as, e.g., open source facial recognition algorithms provided by OpenCV software. For one example, user capture device 307 can capture a facial image of a child that can be a valid passenger of vehicle 100, but deny and prevent the child from driver controls and driving vehicle 100.

For one example, user capture device 307 can be used to capture user images to detect expressions of the user. For example, capture device 307 can capture a selfie, smile, frown, sleeping, dozing, eyes opening and shutting, anger, happiness, sadness, fatigue, anger, stress, or shaking by the user. For one example, the captured expression can be processed and analyzed by computing systems within vehicle 100 in providing a reaction or no reaction is necessary. For example, if capture device 307 captures the user's eyes shutting for a predetermined period of time indicating a driver is falling asleep, a computer for vehicle 100 can react by providing an audio response such as “Tim please wake up you are falling asleep”. Other reactions can include messages on a display, blinking lights on a display, changing settings and preferences, and etc.

Referring to FIG. 3E, for one example, user capture device 307 can include an infrared camera that can capture temperature information 303. Such information can include temperature differential information. For example, a person would have a different temperature differential infrared image than an inanimate object. This type of temperature information 303 can be further used to determine if user head 301 matches a valid user and can detect impersonation. User capture device 307 can be located inside and outside of vehicle 100 and an aligned to capture user images of user head 301. Temperature information can also be compared between users of vehicle 100 to determine wellness of a user in a driver seat or passenger seat and can be used as health data.

### Exemplary Data Processing and Computing System Architecture

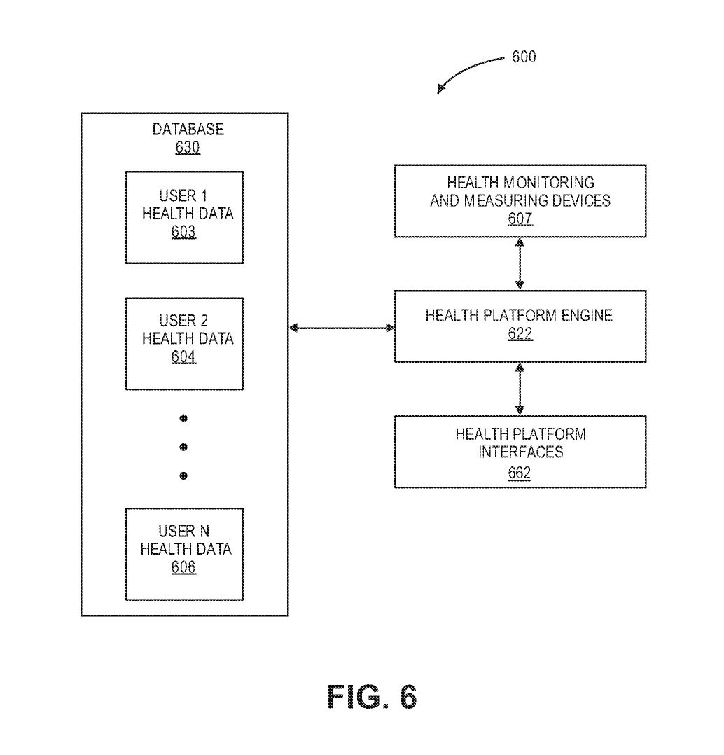

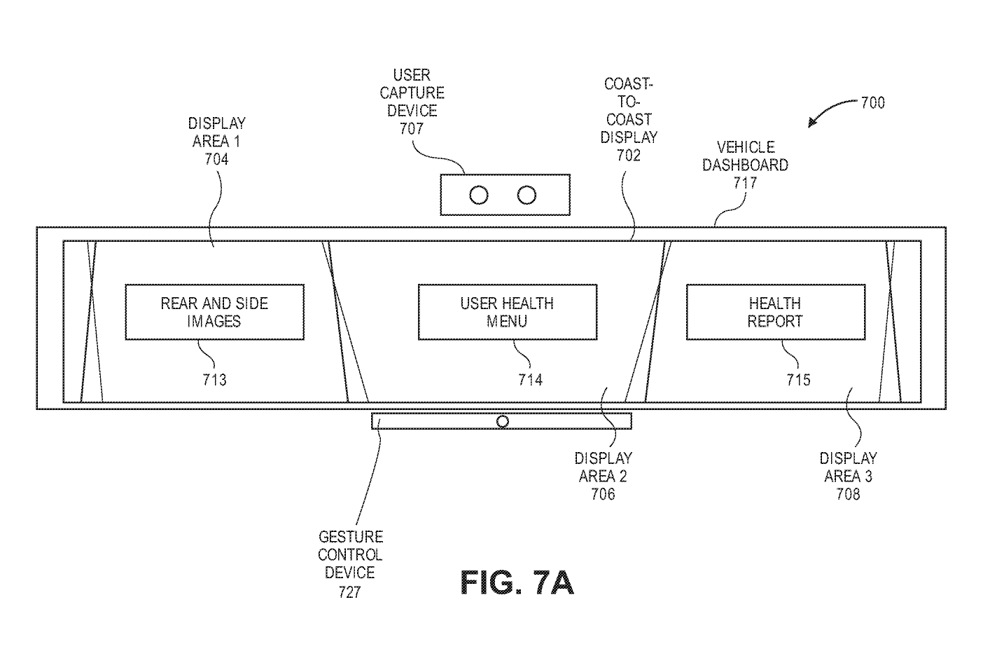

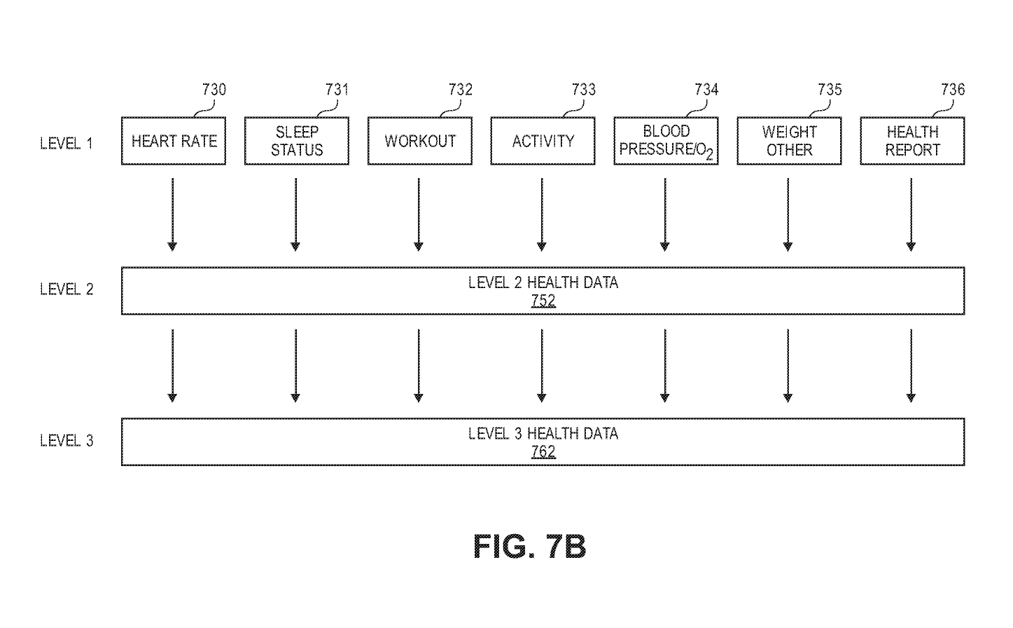

FIG. 4 illustrates one example block diagram of data processing (or computing system) architecture 400 for exemplary environments 100, 200, and 250 of FIGS. 1-2B and 600 and 700 of FIGS. 6-9D. For one example, data processing system architecture 400 can represent a computing or data processing system for vehicle 100 behind or within dashboard 117, 217, 237 in FIGS. 1-2B and dashboard 717 of FIG. 7A. For other examples, architecture 400 can represent a computing system for driver tablet 210 or display computers 101-A and 101-B in FIGS. 1-2B.

Referring to FIG. 4, data processing system architecture 400 includes processor(s) 412, real time operation system 410, and inter-process communication 408 coupled with HMI middleware 402, virtual machine 404, virtual devices(s) environment 406, and secure/online services 414. Processor(s) 412 can include any type of ARM®, nVidia®, or Intel® microprocessor or central processing unit (CPU) configured to perform techniques and operations disclosed herein. For one example, processor(s) 412 can include a system-on-a-chip (SOC) such as nVidia Tegra® providing a graphical processing unit (GPU) architecture which can be used in automobiles providing graphical user interfaces and controls as shown in coast-to-coast display 202 in FIG. 2B and coast-to-coast display 702 in FIG. 7A and FIGS. 9A-9D. For one example, processor(s) 412 can also include nVidia Drive CX hardware and software solutions providing advanced graphics and computer vision navigation for coast-to-coast display 202 and 702 configured to implement techniques and operations disclosed herein. Processor(s) 412 can also include Intel In-Vehicle Infotainment (IVI)® or nVidia Drive CX® processing architecture and software providing information and entertainment features for vehicles configured using techniques and operations disclosed herein.

For one example, real-time operating system 410 can be a Unix® based operating system which can provide cloud connection via security/online services 414 and virtual device communication via virtual device(s) environment 406. Security/online services 414 can include a smart antenna and provide a secure gateway to external cloud services requiring user authentication using high speed wireless communication such as Long-Term Evolution (LTE) standard. Bluetooth communication can also be provided by security/online services 414 for data processing system architecture 400. Virtual devices(s) environment 406 can include, e.g., Android® based environment of devices and virtual machines which can communicate with data processing system architecture 400.

Human machine interface (HMI) middleware 402 can include software to provide graphical user interfaces and controls or a driver or user of environment 100 and driver tablet 110. For one example, HMI middleware 402 can include the Unity® software or Softkinetics® software configured for providing user interfaces and controls to coast-to-coast displays 202 and 702 FIGS. 2B and 7A based on techniques and operations disclosed herein. HMI middleware 402 can also include software to process and analyze captured user images including 2D and 3D facial features, expressions, and temperature information received by user capture devices 107-A through 107-C, 207, 277, 307, and 707 as described in FIGS. 1A-2B, 3C-3D, and 7A. For one example, virtual machine 404 can operate as driver tablet 210 or other computing devices in vehicle 100 or user interface applications for coast-to-coast display 202 or 702 using HMI middleware 402 such as Unity® or Softkinetics® software and inter-process communication 408. HMI middleware 402 can also include software to recognize user gestures from captured by gesture control device 327 using, e.g., Intel Realsense® software and hardware configured to recognize hand gestures to control interfaces on coast-to-coast display 202 or 702.

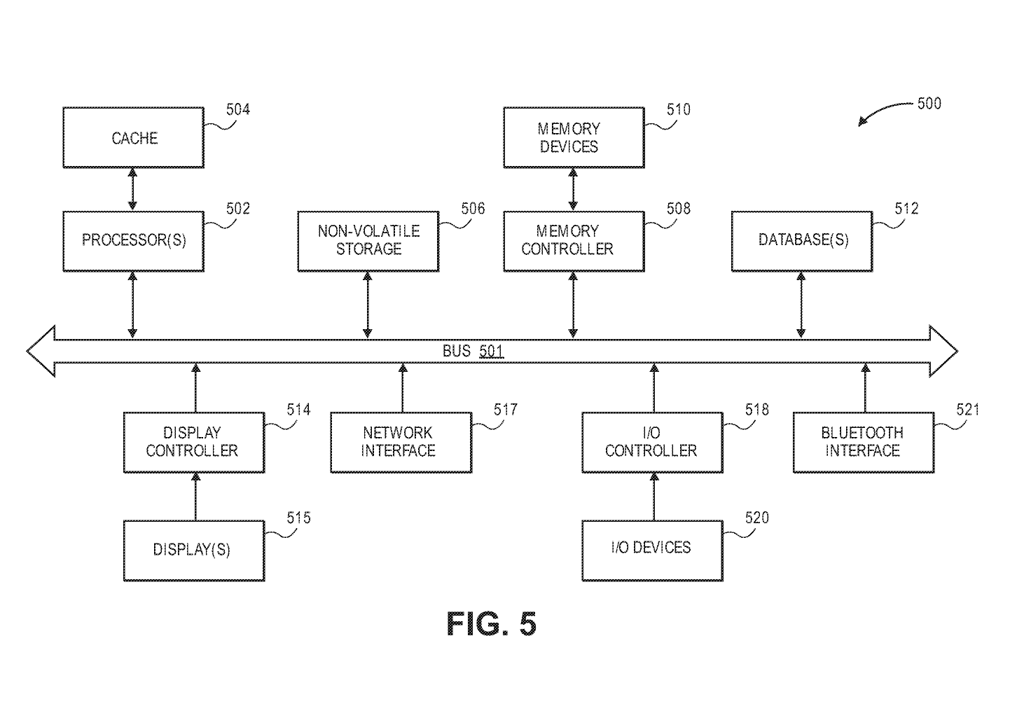

FIG. 5 illustrates one example block diagram of a computing system 500 for the data processing system architecture 400 of FIG. 4. For example, computer system 500 can represent the various components used for a computer such as vehicle computer for vehicle 100 behind or within dashboard 117, 217, 237 and 717 in FIGS. 1-2B and 7A, or driver tablet 210 or display computers 101-A, 101-B. Although FIG. 5 illustrates various components of a data processing or computing system, the components are not intended to represent any particular architecture or manner of interconnecting the components, as such details are not germane to the disclosed examples or embodiments. Network computers and other data processing systems or other consumer electronic devices, which have fewer components or perhaps more components, may also be used with the disclosed examples and embodiments.

Referring to FIG. 5, computing system 500, which is a form of a data processing or computing system, includes a bus 503, which is coupled to processor(s) 502 coupled to cache 504, display controller 514 coupled to a display 515, network interface 517, non-volatile storage 506, memory controller coupled to memory 510, I/O controller 518 coupled to I/O devices 520, and database 512. Processor(s) 502 can include one or more central processing units (CPUs), graphical processing units (GPUs), a specialized processor or any combination thereof. Processor(s) 502 can retrieve instructions from any of the memories including non-volatile storage 506, memory 510, or database 512, and execute the instructions to perform operations described in the disclosed examples and embodiments. Database 512 can also represent a remote databased in a secured-cloud environment in which computing system 500 can communicate with using network interface 517.

Examples of I/O devices 520 include mice, keyboards, printers and other like devices controlled by I/O controller 518. Network interface 517 can include modems, wired and wireless transceivers and communicate using any type of networking protocol including wired or wireless WAN and LAN protocols including LTE and Bluetooth interface and an API interface to communicate with Bluetooth devices. Computing system 500 can also include a Bluetooth interface 521 that provide a Bluetooth interface A separate Bluetooth interface 521 Memory 510 can be any type of memory including random access memory (RAM), dynamic random-access memory (DRAM), which requires power continually in order to refresh or maintain the data in the memory. Non-volatile storage 506 can be a mass storage device including a magnetic hard drive or a magnetic optical drive or an optical drive or a digital video disc (DVD) RAM or a flash memory or other types of memory systems, which maintain data (e.g. large amounts of data) even after power is removed from the system.

For one example, memory devices 510 or database 512 can store health data collected from any number of users (e.g., drivers or passengers) for vehicle 100 using wearable, health measuring devices, user capture devices and sensors. Health data can include bio-scan information such as user images including 2D or 3D facial images and features and expressions. For other examples, memory devices 510 or database 512 can store user information and parameters related to, e.g., MyHealth, MyActivities, or MyEntertainment types of user information for applications on coast-to-coast display 202 and 702. Although memory devices 510 and database 512 are shown coupled to system bus 501, processor(s) 502 can be coupled to any number of external memory devices or databases locally or remotely by way of network interface 517 or Bluetooth interface 521, e.g., database 512 can be secured storage in a cloud environment. For one example, processor(s) 502 can implement techniques and operations described herein for providing a health platform. Display 515 can represent coast-to-coast-displays 202 or 702 or a display for display computers 101-A, 101-B in FIGS. 1-2B and 7A.

Examples and embodiments disclosed herein can be embodied in a data processing system architecture, data processing system or computing system, or a computer-readable medium or computer program product. Aspects, features, and details of the disclosed examples and embodiments can take the hardware or software or a combination of both, which can be referred to as a system or engine. The disclosed examples and embodiments can also be embodied in the form of a computer program product including one or more computer readable mediums having computer readable code which can be executed by one or more processors (e.g., processor(s) 402) to implement the techniques and operations disclosed in FIGS. 1-10.

### Exemplary in-Vehicle Health Platform

FIG. 6 illustrates a block diagram of a computing system 600 to provide an in-vehicle health platform. Computing system 600 includes a memory or database 630 coupled to health platform engine 622. Health platform engine 622 is coupled to health monitoring and measuring devices 607 which can be devices as described in FIGS. 1-3E to receive and collect health data. Health platform engine 622 can store collected health data in database 630 for a plurality of users as user 1 health data (603) to user N health data (606). Users 1-N can be users, drivers or passengers of vehicle 100. Stored health data in database 630 for each user can include sleep data, vital signs data, oxygen level data, and workout data of the user.

Examples of health data stored in database 630 for users 1-N (603-606) can include number of hours awake, light sleep, deep sleep and random eye movement (REM) sleep during each day, week, or month. Examples of vital signs data stored in database 630 can include heart rate in beats per minute (BPM) for each day, week or month including average heart rate data during a period of time, blood pressure data such as diastolic/systolic blood pressure measurements of a user, weight measurements of a user such a weight in lb and kg for each day, week or month. Examples of oxygen level data stored in database 630 can include measurements of a user such as blood oxygen saturation levels during each day, week or month can and provide averages over a period of time such as a week or month. Examples of workout data stored in database 630 can include monitoring data of a physical activity such as walking or running, e.g., number of steps taken during walk or run, time or duration of walk or run, calories burned during activity, etc. for each day, week or month. Such health data can be collected for users 1-N while being monitored in vehicle 100 or retrieved from stored health data for user 1-N in database 630. Database can also store bio-scan user information such as user images and temperature information as described in FIGS. 3D-3E. Database 630 can be a secured storage located within an automobile or computer or in a secured cloud environment accessible by a user.

For one example, health platform engine 622 can implement health platform interfaces 662 as described in FIGS. 7A-9D. Health platform engine 622 can retrieve and collect health data from health monitoring and measuring devices 607 and database 630 and process the health data for users 1-N and provide pattern data and health reports in one or more display areas of coast-to-coast display 202 or 702, e.g., in FIGS. 2B and 7A as shown in FIGS. 8A-9D. Health platform engine can provide menu options for a user to selected the type of pattern and health report to be displayed in vehicle 100 based on, e.g., sleep data, vital signs data, oxygen level data, and workout data of user 1-N. For one example, based on the health data collected and processed, health platform engine 622 can provide user advice or tips to improve fitness or well-being in one or more display areas of a coast-to-coast display or other display in within vehicle 100 as shown, e.g., in FIG. 8C.

### Exemplary in-Vehicle Health Platform Interfaces

FIG. 7A illustrates one example of an in-vehicle health platform (“MyHealth”) user interface 700 on a vehicle dashboard 717. Above dashboard 717 is a user capture device 707 and gesture control device 727 which can operate in the same way as the same devices in FIGS. 1-2B and 3D-3E. Dashboard 717 includes a coast-to-coast display 702 having display areas 1-3 (704, 706, and 708). For one example, display area 1 (704) provides rear and side images 713 of a vehicle (e.g., vehicle 100). For example, as shown in FIG. 8A, display area 1 (704) shows rear and side images 713 from cameras situated on the vehicle to take images of vehicles behind and to the side of the vehicle. Display area 1 (704) can also show an image of the driver (“Lucas”) and temperature along with settings and controls at the top of display area 1 (704). For this example, the driver Lucas can see the rear and side view images in display area 1 (704).

Display area 2 (706) of coast-to-coast display 702 on dashboard 717 can provide a user health menu 714. For example, as shown in FIG. 8B, display area 2 (706) can show a MyHealth option menu for a user. The menu includes options for Sleep, Vital Signs, Workout, and O₂ level. For one example, a user hand can select one of the options captured by gesture control device 727. If an option is selected, further information, e.g., pattern information and health reports can be provided based on the selected health data option as described in FIGS. 7B and 9A-9D. For one example, next to each option, current or last health data measurements can be provided or displayed such as number of hour slept (e.g., 8.3 hours), heart rate (82 BPM), blood pressure 138/98 diastolic/systolic, workout time (e.g., 0.3 hours), and oxygen level (92%). Other measurement data can be provided or displayed to the right of display area 2 (706) such as miles walked (e.g., 2.1 miles), steps walked (e.g., 4,205 steps), calories burned (e.g., 221 calories), and weight (e.g., 185 lbs). Such measurements can be provided by wearables, health measurement devices, sensors etc. as described herein.

Display area 3 (708) of coast-to-coast display 702 on dashboard 717 can provide a health report 715 for a user. For example, as shown in FIG. 8C, display area 3 (708) can provide a health report providing advice and tips to improve health and well-being having topics such as Fulfillment, Clear Mind, and Strong Heart. For example, under Fulfillment the report can provide content: “Great job in achieving 6,300 steps. To complete your daily goal of 10,000 steps, arrive earlier at your next appointment, park your car a mile away then walk.” An option such as +Add to Navigation can be provided to enter the location of the next appointment into a navigation application for the vehicle. Under Clear Mind, for example, the health report can provide content: “Your last measured oxygen blood level was lower than normal. Spend some time outside with your kids to improve your blood oxygen level.” For this suggestion, a +Set Reminder option can be provided to set a reminder in a calendar application for the vehicle. Under the Strong Heart, for example, the health report can provide content: “Your resting heart rate and blood pressure are slightly higher than normal. Increasing your daily workout +1 hours improves blood pressure and heart health.” For this suggestion, a + Add to Calendar option can be provided schedule a daily workout for the user to a calendar application.

FIG. 7B illustrates one example of multiple level health data processing for the health platform user interface of FIG. 7A. For one example, three levels of health data for processing are shown (e.g., levels 1-3). At level 1, health data categories or options include heart rate 730, sleep status 731, workout 732, activity 733, blood pressure/O2 734, weight other 735, and health report 736. If one of these categories or options are selected by a user in the MyHealth platform, level 2 and 3 processing can be performed providing different levels of health data 752 and 753 to the MyHealth platform interface. For example, each level of health data can be provided in separate interfaces or displays to the user.

For one example, level 2 health data 752 for heart rate 730 can be real time measurement of the actual heart rate can be measured and displayed along with, e.g., daily minimum and maximum heart rate values. Level 3 health data 762 for heart rate 730 can provide or display details of daily, weekly or monthly statistics can be provided along with patterns for the heart rate. For sleep status 731, level 2 health data 752 can provide or display the sleep pattern of the last night sleep for the user. Level 3 health data 762 can provide or display a detailed explanation of the different sleep phases or the user and daily, weekly or monthly statistics.

For workout 732, level 2 health data 752 can provide or display a summary of the last workout for the user. Level 3 health data 762 for workout 732 can provide or display a more detailed explanation of the heart rate with explanation of the meaning of different heart rate intervals during the workout. For activity 733, level 2 health data 752 can provide or display a summary of the accomplished steps distance and burned calories of the current day. Level 3 health data 762 for activity 733 can provide or display further details containing the relevant daily and weekly statistics of accomplished steps, distance and calories burned. For blood pressure/SpO₂ 734, level 2 health data 752 can provide or display the measured blood pressure value and oxygen saturation levels. Level 3 health data 762 for blood Pressure/SpO₂ 734 can provide or display further details, e.g., overview of the last performed measurements or daily, weekly, or monthly statistics.

For weight other 735, level 2 health data can provide or display the measured weight. Other data can be displayed or provided such as mood and fatigue with and indicator or score using images of the user of face tracking by user capture devices disclosed herein. Level 3 health data 762 for weight other 735 can provide of display obtained through further details of weight variation during the last day, week, or months or mood or fatigue variations. For health report 736, level 2 health data can provide or display a consolidated health report for each user based on all the health data generated and provided the health platform. This information can provide valuable and important advice and tips to improve the health and well-being of a user. Level 3 health data 762 for health report 736 can provide further details and explanation of advice and tips including providing options to act on the advice and tips such as scheduling workouts and navigating to locations for obtaining desired number of steps and distances to walk or run.

FIG. 9A illustrates one example of user sleep data provided on the health platform user interface. The interface of FIG. 9A can include level 2 and 3 health data as disclosed in FIG. 7A for a MyInsight window for Sleep. For one example, sleep patterns for a user can be shown for today in a 24-hour window from Sam to Sam are shown showing amount of time spent awake (e.g., 0 hours awake), light sleep (e.g., 3.5 hours), REM sleep (e.g., 2.5 hours), and deep sleep (e.g., 2.3 hours). Sleep patterns can also be shown weekly or monthly which can be selected by a user. On the right of the window, other health data and graphical elements can be displayed such a sleep meter indicating total number of hours slept for the day (e.g., 8.3 hours).

FIG. 9B illustrates one example of user vital signs data provided on the health platform user interface. The interface of FIG. 9B can include level 2 and 3 health data as disclosed in FIG. 7A for MyInsight window for Vital Signs. For one example, the window for Vital Signs displays weekly patterns of systolic blood pressure (sbp), diastolic blood pressure (dpb), heart rate in beats per minute (bpm). Alternatively, daily patterns or weekly patterns can be displayed. Referring to FIG. 9B, minimum and maximum sbp indicators are displayed (e.g., 118 sbp Min, 158 sbp Max) and minimum and maximum dbp indicators are displayed (e.g., 76 dbp Min, 91 dbp Max) at the top of the window. Patterns blood pressure mmHG measurement ranges are shown from Monday through Sunday from 60 to 200. On the right of the window, other information and graphical elements can be displayed such as the current heart rate indicator (e.g., 82 bpm) ranging from 68 to 110 with a previous heart rate measurement (e.g., 44 bmp) and a weekly average heart rate measurement (e.g., 78 bpm).

FIG. 9C illustrates one example of user workout data provided on the health platform user interface. The interface of FIG. 9C can include level 2 and 3 health data as disclosed in FIG. 7A for Mylnsight window for Workout. For one example, Workout window can provide the same or similar heart rate and blood pressure data has FIG. 9B, e.g., 118 sbp Min, 158 sbp Max, 76 dbp Min, 91 dbp Max, and 82 beats per minute. For one example, workout data patterns can be shown for today or, alternatively, patterns can be shown weekly or monthly. In this window, workout activity is shown for walking between 8 am to 10 am, running from 6 pm to 8 pm, cycling from 6 pm to 8 pm and yoga from 4 pm to 6 pm. On the right of the window, other information and graphical elements can be displayed such as total distance walked or during running (e.g., 8.5 miles) and calories burned 1,155 calories.

FIG. 9D illustrates one example of user oxygen level data provided on the health platform user interface. The interface of FIG. 9D can include level 2 and 3 health data as disclosed in FIG. 7A for Mylnsight window for Oxygen Level. For one example, Workout window can provide the same or similar heart rate and blood pressure data has FIGS. 9B-9C, e.g., 118 sbp Min, 158 sbp Max, 76 dbp Min, and 91 dbp Max. For one example, oxygen level patterns can be shown for the week from Monday through Sunday showing oxygen levels between 80% and 100% today or, alternatively, oxygen level patterns can be shown daily or monthly. On the right of the window, other information and graphical elements can be displayed such current oxygen level measurement (e.g., 96%) and previous oxygen level measurement (e.g., 92%) and what is considered a healthy range, e.g., 96-99%. For the examples of FIGS. 9A-9D, the health data patterns can be based on other types of health data which can be collected and added to the Mylnsight windows such as weight measurements. The Mylnsight windows can be displayed in one of the display areas of a display and another display area can display health reports related to the patterns shown in FIGS. 9A-9D as described in FIG. 8C. The health platform can display patterns as charts or graphs with any number of graphical elements and indicators as described herein.

### Exemplary in-Vehicle Health Platform Methods

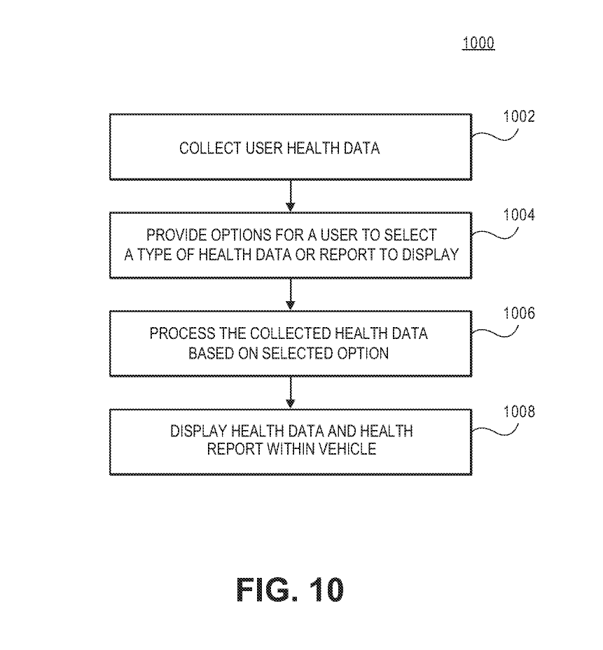

FIG. 10 illustrates one example of a flow diagram 1000 of a process to provide in-vehicle user health data and reporting. Flow diagram includes blocks 1002 through 1008. At block 1002, health data for a user is collected. For one example, health data can be collected from a wearable, health measuring devices, or sensors and user capturing devices that measure health data for the user (e.g., wearable 150, 250, 350; health measuring devices 152, 252, 352-1, 352-2; seats with weight sensors 128-A and 128-B, weight sensors 343; user capture devices 107-A to 107-C, 207, 277, 307). Alternatively, health data can be collected from health data stored in a database (e.g., database 630) which can be located in a secured cloud-based environment or located within the vehicle (e.g., vehicle 100).

At block 1004, options are provided for the user to select a type of health data and report to display based on the collected health data. For one example, a MyHealth option can be provided in a display area of a cost-to-coast display area (e.g., rotating polyhedron 217 in display area 2 (206) that provides a menu of health data options for Sleep, Vital Signs, Workout and O₂ level (e.g., display area 3 (208)). For one example, if a user selected the MyHealth application, MyHealth menu as shown in FIG. 8B can be provided in a display area of a display within the vehicle for a user to select an option to display related health data or report for a user. For example, if a user selects one of the options, a health report as shown in FIG. 8C can be provided or displayed in a display area of a display of the vehicle. Additionally, health data patterns and statistics as shown in FIGS. 9A-9D can be displayed in one or more display areas of the display of the vehicle.

At block 1006, the collected health data is processed data based on the selected options. For one example, a computer, computing system or data processing system (e.g., computer or computing system 400, 500, and 600) can process health data in a database (e.g., database 630) or health data sources to determine health patterns and statistics and relative to a period of time over, e.g., a day, week, or month and by hours of the day as shown in FIGS. 9A-9D. For one example, data models or machine learning models can be applied to the collected to determine advice and tips to provide to a user in health reports as shown in FIG. 8C. Other examples can include decision trees or table matching of advice and tips to health data ranges in determining advice and tips to provide in the health reports.

At block 1008, the selected type of health data and health report are displayed in one or more display areas of a display within the vehicle based on the processed collected health data. For example, patterns of health data as shown in FIGS. 9A-9D can be displayed as well as a health report as shown in FIG. 8C.

In the foregoing specification, the invention has been described with reference to specific exemplary embodiments thereof. It will, however, be evident that various modifications and changes may be made thereto without departing from the broader spirit and scope of disclosed examples and embodiments. The specification and drawings are, accordingly, to be regarded in an illustrative rather than a restrictive sense.

What is claimed is:

1. An in-vehicle health platform system comprising:
a display within a vehicle, the display having a plurality of display areas; and
a computer coupled to the display, the computer to receive health data for a user and to provide options for the user to select a type of health data and report to display in one or more display areas of the display based on the received health data.

2. The in-vehicle health platform system of claim 1, wherein the health data includes at least one of sleep data, vital signs data, oxygen level data, and workout data of the user.

3. The in-vehicle health platform system of claim 2, wherein the computer is to receive health data from at least one of a wearable, a health measuring device, and a weight measuring device.

4. The in-vehicle health platform system of claim 3, wherein the wearable is worn by the user, the health measuring device is mounted within the vehicle, and the weight measuring device includes one or more sensors in a seat of the vehicle.

5. The in-vehicle health platform system of claim 2, wherein the menu options include sleep option to display or report the sleep data, vital signs option to display or report the vital signs data, oxygen level option to display or report the oxygen level data, and workout option to display or report the workout data.

6. The in-vehicle health platform system of claim 5, wherein the computer is to display or report the health data by displaying patterns of the health data for the user related to at least one of the sleep option, vital signs option, oxygen level option and workout option in one or more display areas of the display.

7. The in-vehicle health platform system of claim 6, wherein the patterns include one or more graphical elements shown on the display.

8. The in-vehicle health platform system of claim 7, wherein the computer is to provide user advice or tips to improve fitness or well-being in one or more display areas of the display based on the patterns of the health data.

9. The in-vehicle health platform system of claim 8, wherein the advice or tips include activity suggestions to achieve health or fitness goals or health measurement levels for the user.

10. The in-vehicle health platform system of claim 1, further comprising a database to store the health data for the user, wherein the database is located in a secured cloud-based system.

11. The in-vehicle health platform system of claim 1, wherein the display is on a dashboard of the vehicle or a display computer.

12. A computer-implemented method within a vehicle, the method comprising:
collecting health data for a user;
providing options for the user to select a type of health data and report to display based on the collected health data;
processing the collected health data based on the selected options; and
displaying the selected type of health data and health report in one or more display areas of a display within the vehicle based on the processed collected health data.

13. The computer-implemented method of claim 12, wherein collecting health data includes collecting health data from a wearable, a health measuring device, or a weight measuring device related to a user.

14. The computer-implemented method of claim 13, further comprising collecting sleep data, vital signs data, oxygen level data, or workout data related to the user from at least one of the wearable, health measuring device, or weight measuring device within the vehicle.

15. The computer-implemented method of claim 14, further comprising receiving health data from wearable, health measuring device, or weight measuring device via a Bluetooth or WiFi communication protocol.

16. The computer-implemented method of claim 14, further comprising:
selecting one of a sleep option, vital signs option, oxygen level option, and workout option for the user; and
displaying one of health data or health report of sleep data, vital signs data, oxygen level data, and workout data based on the selected option for the user in one or more display areas of the display.

17. The computer-implemented method of claim 16, wherein displaying one of health data or health report includes displaying patterns of one of the sleep data, vital signs data, oxygen level data, and workout data based on the selected option for the user in one or more display areas of the display.

18. The computer-implemented method of claim 16, wherein displaying one of health data or health report includes displaying user advice or tips to improve fitness or well-being based on the selected option for the user in one or more display areas of the display.

19. The computer-implemented method of claim 18, wherein displaying user advice or tips includes displaying suggestions to achieve health or fitness goals or health measurement levels for the user.

20. The computer-implemented method of claim 12, further comprising storing the collected health data in a database for the user, wherein the database is located in a secured cloud-based system.

21. The computer-implemented method of claim 20, further comprising:
downloading the collected health data for the user from the database; and
generating health data or health report for display based on the downloaded health data from the database.

22. A non-transitory computer-readable medium, including instructions which if executed by a computer, cause the computer to:
collecting health data for a user;
providing options for the user to select a type of health data and report to display based on the collected health data;
processing the collected health data based on the selected options; and
displaying the selected type of health data and health report in one or more display areas of a display within the vehicle based on the processed collected health data.

23. The non-transitory computer-readable medium of claim 22, including instructions which if executed by the computer, cause the computer to collect health data from a wearable, a health measuring device, or a weight measuring device related to a user.

24. The non-transitory computer-readable medium of claim 23, including instructions which if executed by the computer, cause the computer to collect sleep data, vital signs data, oxygen level data, or workout data related to the user from at least one of the wearable, health measuring device, or weight measuring device within the vehicle.

25. The non-transitory computer-readable medium of claim 23, including instructions which if executed by the computer, cause the computer to receive health data from the wearable, health measuring device, or weight measuring device via a Bluetooth communication protocol.

26. The non-transitory computer-readable medium of claim 24, including instructions which if executed by the computer, cause the computer to
select one of a sleep option, vital signs option, oxygen level option, and workout option for the user; and
display one of health data or health report of sleep data, vital signs data, oxygen level data, and workout data based on the selected option for the user in one or more display areas of the.

27. The non-transitory computer-readable medium of claim 26, including instructions which if executed by the computer, cause the computer to display patterns of one of the sleep data, vital signs data, oxygen level data, and workout data based on the selected option for the user in one or more display areas of the display.

28. The non-transitory computer-readable medium of claim 24, including instructions which if executed by the computer, cause the computer to display user advice or tips to improve fitness or well-being based on the selected option for the user in one or more display areas of the display.

29. The non-transitory computer-readable medium of claim 28, including instructions which if executed by the computer, cause the computer to display suggestions to achieve health or fitness goals or health measurement levels for the user.

30. The non-transitory computer-readable medium of claim 22, including instructions which if executed by the computer, cause the computer to store the collected health data in a database for the user, wherein the database is located in a secured cloud-based system.

31. The non-transitory computer-readable medium of claim 24, including instructions which if executed by the computer, cause the computer to
download the collected health data for the user from the database; and
generate health data or health report for display based on the downloaded health data from the database.
